(19) United States
(12) Patent Application Publication
(10) Pub. No.: US 2019/0353926 A1
(43) Pub. Date: Nov. 21, 2019

(54) VERGENCE DETECTION METHOD AND SYSTEM

(71) Applicant: Johnson & Johnson Vision Care, Inc., Jacksonville, FL (US)

(72) Inventors: Adam Toner, Jacksonville, FL (US); Donald K. Whitney, Melbourne, FL (US)

(21) Appl. No.: 15/979,612

(22) Filed: May 15, 2018

(51) Int. Cl. G02C 7/02; G02C 7/08; A61B 5/00

(52) U.S. Cl. CPC G02C 7/027; A61B 2562/046; A61B 5/6803; G02C 7/081

(57) ABSTRACT

A vergence detection system is incorporated into an ophthalmic lens to automatically determine if the lens wearer is trying to accommodate by viewing a near object or gazing into the distance to view a far object by measuring the vergence angles as the wearer is trying to see near or far. The vergence detection system utilizes multiple sensors to measure certain parameters and make a calculation to determine vergence.

### I. FIELD OF INVENTION

The present invention relates to ophthalmic lenses having embedded elements, and more specifically, to use the embedded elements to automatically determine if the lens wearer is trying to accommodate or not, by measuring the vergence angles as the user is trying to converge or diverge.

### II. DISCUSSION OF THE RELATED ART

Near and far vision needs exist for all. In young non-presbyopic patients, the normal human crystalline lens has the ability to accommodate both near and far vision needs, and those viewing items are in focus. As one ages, the vision is compromised due to a decreasing ability to accommodate. This is called presbyopia.

Adaptive optics products and their use are positioned to address this and restore the ability to see items in focus. But what is required is knowing when to “activate/actuate” the optical power change. A manual indication or use of a key fob to signal when a power change is required is one way to accomplish this change. However, leveraging anatomical/biological conditions/signals may be more responsive, more user friendly and potentially more “natural” and thus more pleasant.

A number of things happen when a person changes his/her gaze from far to near. The pupil size changes and the line of sight from each eye converge in the nasal direction coupled sometimes with a somewhat downward component as well. However, to sense/measure these items is difficult, one also needs to filter out certain other conditions or noise, (e.g.: blinking, what to do when one is lying down, or head movements).

At a minimum, sensing of multiple items may be required to remove/mitigate any false positive conditions that would indicate a power change is required when that is not the case. Additionally, threshold levels may vary from patient to patient, thus some form of calibration and/or customization may be beneficial as well.

### III. SUMMARY OF THE INVENTION

In at least one embodiment, a user-wearable ophthalmic lens includes: a plurality of sensors; a signal-processing unit in communication with the plurality of sensors and configured to receive sensor signals from the plurality of sensors; a noise-rejection unit in communication with the signal-processing unit and configured to receive signal-processed signals from the signal-processing unit; and a decision-making unit in communication with the noise-rejection unit and configured to receive corrected, processed signals from the noise-rejection unit, the decision-making unit configured to change accommodation of the user-wearable ophthalmic lens based on the processed signals.

In a further embodiment to the previous embodiment, calibration of the at least one ophthalmic lens is initiated upon receipt of a calibration signal from an external device. In a further embodiment to the previous embodiment, the plurality of sensors, the signal-processing unit, the noise-rejection unit, and the decision-making unit are configured to determine a customized vergence angle threshold. In a further embodiment to either embodiment of this paragraph, the external user device is a smartphone.

In a further embodiment to the previous embodiments, the signal-processing unit, the noise-rejection unit, and the decision-making unit use a customized vergence angle threshold to determine if there is a need to change accommodation.

In at least one embodiment, a system includes: a pair of ophthalmic lenses, each lens having a system controller; a plurality of sensors having a six-axis array to supply sensor signals to the system controller; and a lens activator configured to receive control signals from the system controller, and where at least one of the system controllers determining a vergence angle for the lenses based on at least signals from the plurality of sensors in the six-axis sensor array per lens and controlling a change in accommodation of at least the lens on which the system controller is located. In a further embodiment to the previous embodiments, the system controller in each lens using the plurality of sensors calculates the eye yaw of each eye and then shares the information to calculate the difference of each eye yaw to determine the total vergence angle of the wearer. In a further embodiment to the other embodiments of this paragraph, the six-axis sensor array includes at least one of a combination of an accelerometer and magnetometer for X-axis, a combination of an accelerometer and magnetometer for Y-axis, and a combination of an accelerometer and magnetometer for Z-axis.

In at least one embodiment, a method for determining vergence angle using two ophthalmic lenses, each having a plurality of sensors, a lens activator, and a system controller includes: generating a plurality of sensor signals from the plurality of sensors for at least one of the system controllers; setting a vergence angle for the lenses by at least one system controller based on the plurality of sensor signals from the plurality of sensors; generating a control signal to change accommodation level by the at least one system controller for the lens activators after the vergence angle has crossed a predetermined vergence angle threshold; and changing the accommodation levels of the lenses by the respective lens activator in response to the control signal.

In a further embodiment to the previous method embodiment, the plurality of sensors in each lens is a two-axis sensor array; and the setting the vergence angle is done by both system controllers using the sensor signals from the respective 2-axis sensor array by calculating an eye yaw difference, and the method further includes sharing the set vergence angle between the system controllers through a communication link. In a further embodiment to the method embodiment of the previous paragraph, the plurality of sensors in each lens is a two-axis sensor array; and the setting the vergence angle is done by both system controllers using the sensor signals from the respective two-axis sensor array, and the method further comprising sharing the set vergence angle between the system controllers through a communication link.

In a further embodiment to the first method embodiment, the plurality of sensors in each lens is a three-axis sensor array; and the setting the vergence angle is done by both system controllers using the sensor signals from the respective three-axis sensor array by calculating an eye yaw difference, and the method further includes sharing the set vergence angle between the system controllers through a communication link. In a further embodiment to the first method embodiment, the plurality of sensors in each lens is a three-axis sensor array; and the setting the vergence angle is done by both system controllers using the sensor signals from the respective three-axis sensor array, and the method further comprising sharing the set vergence angle between the system controllers through a communication link.

In a further embodiment to the first method embodiment, the plurality of sensors in each lens includes a multi-axis sensor array, and the method further comprising comparing with the system controller the total signal of each multi-axis sensor array to a known level representing the total acceleration of gravity; and rejecting the sensor signals when the total signal is out of range. In a further embodiment to the first method embodiment, the plurality of sensors in each lens includes a multi-axis sensor array where the axes are offset from measurement nulls such that the measurement axis is perpendicular to a vector representing gravity.

In a further embodiment to any of the previous embodiments, the sensor array includes an accelerometer for an X-axis and a second accelerometer for a Y-axis, a magnetometer for an X-axis, and/or a magnetometer for a Y-axis.

In a further embodiment to any of the previous embodiments, the ophthalmic lenses are either contact lenses or intraocular lenses.

### IV. BRIEF DESCRIPTION OF THE OF THE DRAWINGS

The foregoing and other features and advantages of the disclosure will be apparent from the following, more particular description of preferred embodiments of the disclosure, as illustrated in the accompanying drawings.

FIG. 1 illustrates an example of focus determination.

FIG. 2 illustrates an example implementation according to an embodiment of the present invention.

FIG. 3 illustrates a flowchart according to an embodiment of the present invention.

FIGS. 4A-4C illustrate example implementations according to embodiments of the present invention.

FIG. 5 illustrates another example implementation according to an embodiment of the present invention.

FIG. 6 illustrates a flowchart according to another embodiment of the present invention.

### V. DETAILED DESCRIPTION

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting. As will be appreciated by one skilled in the art, aspects of the present invention may be embodied as a system, method or computer program product.

Because everyone's eyes are a bit different, (e.g. pupil spacing and location, lens-on-eye position, etc.), even at a fixed close distance, initial vergence angles will differ from wearer to wearer. It may be useful once the lenses are placed on (or in) the eye to calibrate what the initial vergence angle is, so that differences in this angle can be assessed while in service. This value can be used for subsequent vergence calculations.

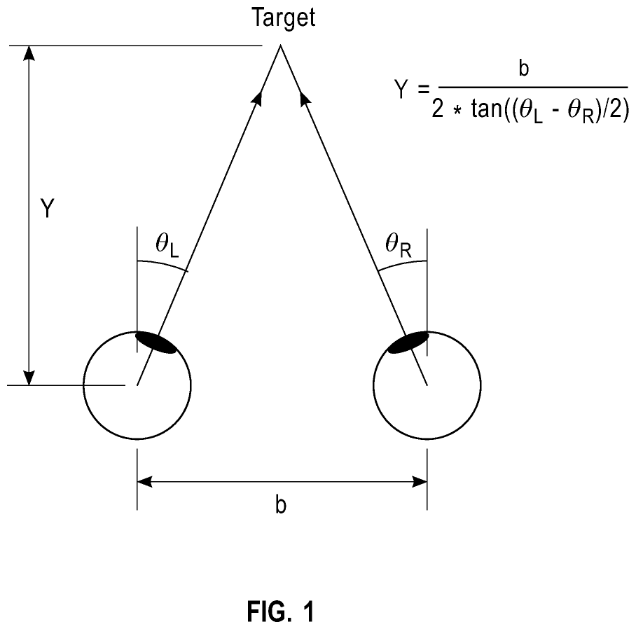

In reference to FIG. 1, when observing an object, in each eye the visual axis points toward the object or Target. Since the two eyes are spaced apart (distance b) and the focal point is in front, a triangle is formed. Forming a triangle allows the relationship of angles (θL and θR) of each visual axis to the distance (Y) to the object or Target is from the eyes to be determined. Since the distance (Y) is what determines if a change in optical power is required, then knowing the angles and the distance between the eyes and using simple math would allow a system to make a decision regarding when to change the optical power. The equation to calculate the distance Y is given by Y=b/(2*tan((θL−θR)/2)).

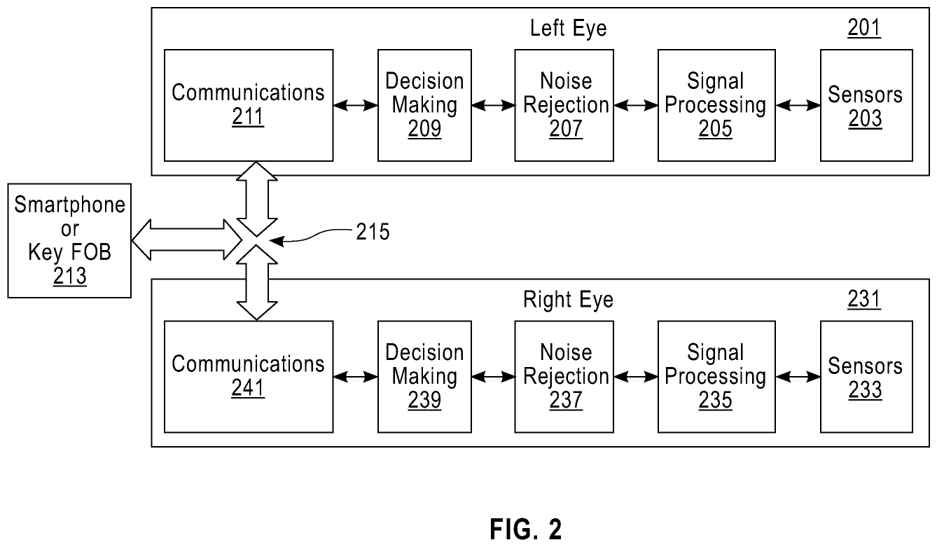

FIG. 2 illustrates an exemplary system according to an embodiment of the present invention. A pair of user-wearable ophthalmic lenses 201, 231 that each includes a plurality of embedded elements including at least one sensor 203, 233, a signal-processing unit 205, 235, a noise-rejection unit 207, 237, a decision-making unit 209, 239, and a communication unit 211, 241, respectively. For this disclosure, ophthalmic lenses include both contact lenses (e.g., daily disposables or reusable contact lenses) and intraocular lenses. Sensors 203, 233 provide pupil eye movement signals to a signal processing unit 205, 235. The processed signal is provided to a noise-rejection unit 207, 237. The corrected processed signals are sent to a decision-making unit 209, 239. As described hereafter, the decision-making unit 209, 239 compares the measured vergence angle against the threshold and determines if any changes in lens accommodation are necessary. The decision-making unit 209, 239 communicates with a communication unit 211, 241 which in turns allows for communication to either a smartphone or key fob 213. The decision-making unit 209, 239 can further communicate with the other lens through communication channel 215. The decision-making unit 209, 239 may comprise any suitable device for calculating and comparing, such as a microprocessor. Additional functionality and embodiments for the system are described hereafter.

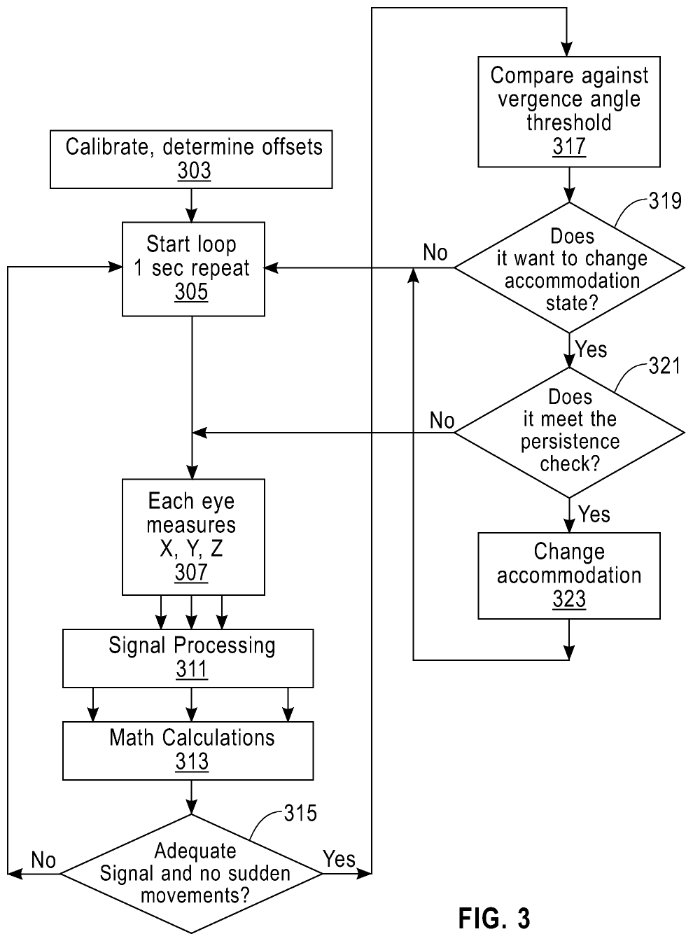

FIG. 3 illustrates a method according to an embodiment of the present invention. There are two questions the system will ask in at least one embodiment: A) “Are the conditions suitable for making a decision?” and B) “Should the system activate or deactivate the lens?” The decision making starts with the question—“Are the conditions suitable for making a decision?” Between the filtering and other indicators, the accommodation scheme determines if there is a sufficient signal or that the conditions are acceptable to make a decision on question B. If it is determined that if the system cannot make a good decision based on a good signal, then it is better for the user not to allow a change in states, but to stay in the current state. This would be preferable to erratically changing from state to state or to change unexpectedly from one state to the other. The nulls are places where the signal is too weak to calculate the signal accurately. Other occurrences that confound measurement also include identifying blinks, where the signal is unstable, and sudden accelerations that are not consistent with wanting to change the accommodation mode. If “the conditions suitable for making a decision,” then the system must compare the current reading against a threshold. This could involve persistence checking. The system is always looking for the correct conditions to allow measurement and then the correct conditions for a change in accommodation. In at least one embodiment, the system measures the convergence angles of the two eyes and calculates the difference and then compares it to the preset threshold. The threshold will have hysteresis, and can be determined in the doctor's office, in the factory, or even during calibration using a smartphone application (or app).

Still referring to FIG. 3, the process continues with conducting a calibration and determining offsets 303. Once the calibration and determining offsets process is done, the process conducts an on-going vergence analysis loop 305. This vergence analysis loop is repeated at, for example, one-second intervals. A first process after the start of the loop is to measure each eye position, relative to gravitational pull or the magnetic field of the earth, along each X, Y, Z axis 307. These measured signals are submitted to signal processing 311 and processed signals are subjected to mathematical calculations 313. The process analyzes if the signal, as a result of the mathematical calculations, is adequate and there are no sudden eye movements 315. If the signal is not adequate (NO), then the process restarts the loop 305. If the decision signal is adequate (YES), then the movement signal is compared to a vergence angle threshold 317. A determination is made to see if there should be a change in the accommodation state 319. If no change in the accommodation state is needed (NO), then the loop restarts again 305. If there is a need to change the accommodation state (YES) the process conducts a persistence check 321. If the persistence check 321 fails (NO), then the loop restarts again 305. If the persistence check 321 is good (YES) then the process changes the accommodation 323 by physically using lens activators. Once the accommodation has been changed, then the overall loop restarts again 305.

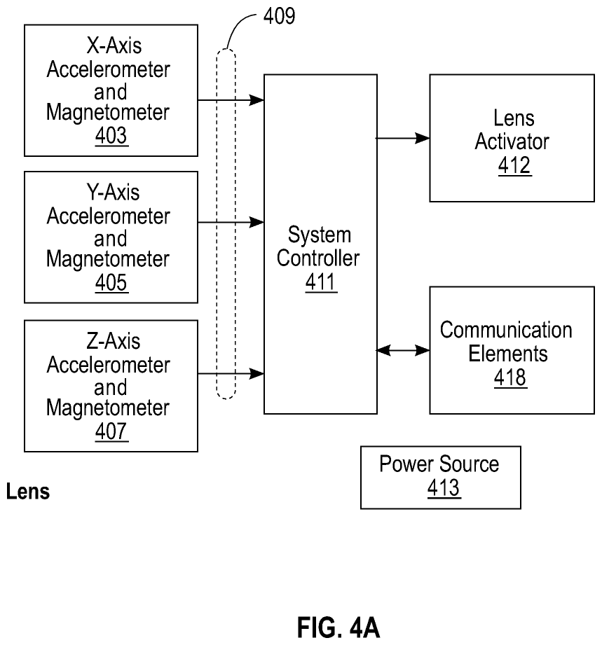

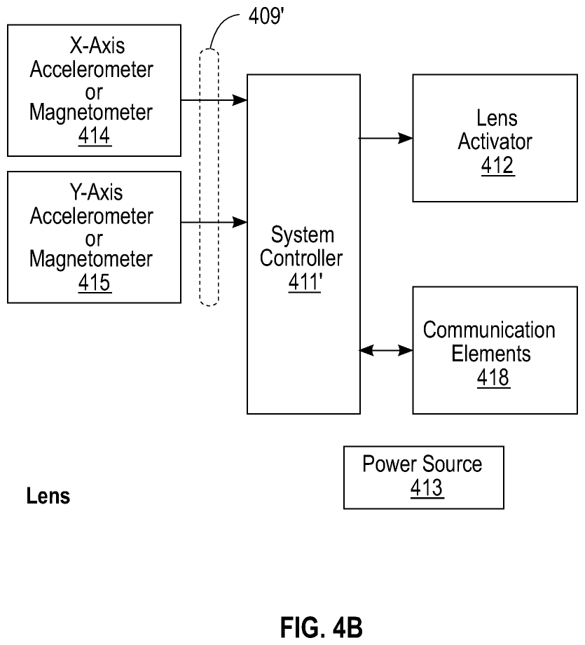

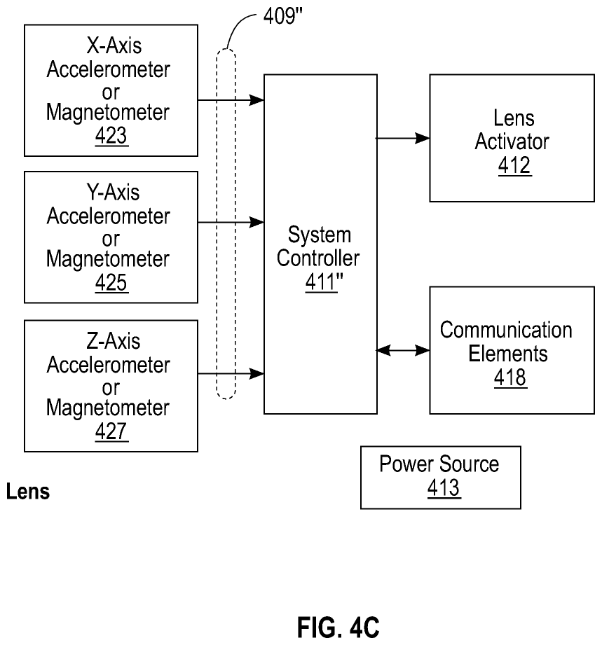

In a six-axis system, similar to an aircraft system, there are X axis, Y axis and Z axis accelerometer sensors and there are three (X, Y, Z) magnetometer sensors as shown in FIG. 4A described in detail subsequently. Using Euler Rotational Matrices to compensate for rotated frames of reference, angles of direction may be accurately determined. These angles are usually referred to as pitch, roll, and yaw. The direction of each eye may be determined using these sensors. If each eye has all six sensors on a lens, the yaw for each eye or (θL and θR) from FIG. 1 can be determined. The general assumption is that the pitch and roll of each eye are very close to the same on the left and right eyes. The yaw difference could be used to determine the vergence angle and, using the equation in FIG. 1, relate it to the distance between the lens and the Target or object of focus. This information would then be tested against the threshold. The six sensors are used to accurately calculate the yaw angle. Once both sets of values are available, the difference may be calculated and compared to the threshold to see if the wearer is trying to accommodate.

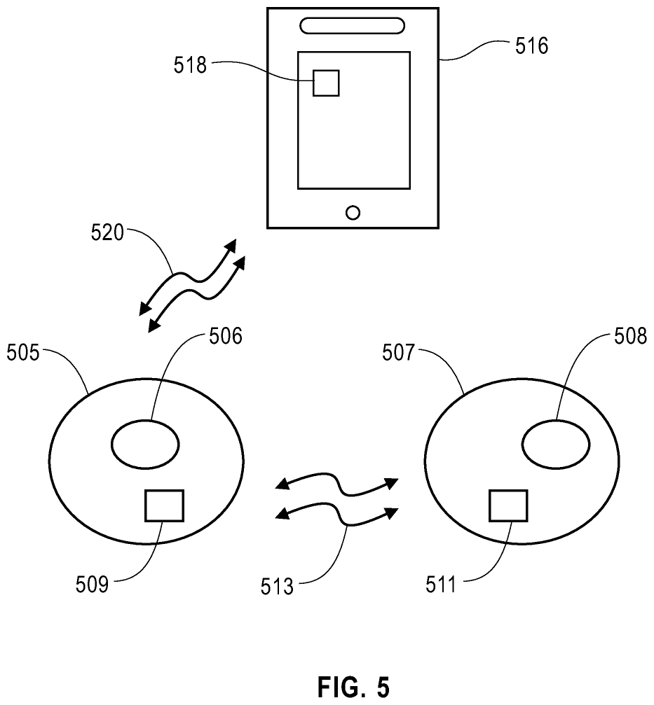

FIG. 4A illustrates a six-axis system according to an exemplary embodiment of the present invention. A system controller 411 controls a lens activator 412 that changes the adaptive optics (see FIG. 5) to control the ability to see both near and far items in focus. The system controller 411 receives control signals 409 from a plurality of multidimensional sensors. A first multidimensional sensor includes an X-axis accelerometer and magnetometer 403. A second multidimensional sensor includes a Y-axis accelerometer and magnetometer 405. A third multidimensional sensor includes a Z-axis accelerometer and magnetometer 407. The system controller 411 further receives from and supplies signals to communication elements 418. Communication elements 418 allow for communications between the user lens(es) and other devices such as a near-by smartphone. The system controller 411 may include the other elements as described with reference to FIG. 2.

A power source 413 supplies power to all of the lens components (or elements). The power source 413 may be a fixed power supply, wireless charging system, or rechargeable power supply elements. The power may be supplied from a battery, a primary cell, an energy harvester, or other suitable means as is known to one of ordinary skill in the art. Essentially, any type of power source 413 may be utilized to provide reliable power for all other components of the system. In an alternative embodiment, communication functionality is provided by an energy harvester that acts as the receiver for the time signal, for example, in an alternative embodiment, the energy harvester is a solar cell, a photovoltaic cell, a photodiode, or a radio frequency (RF) receiver, which receives both power and a time-base signal (or indication). In a further alternative embodiment, the energy harvester is an inductive charger, in which power is transferred in addition to data such as RFID. In one or more of these alternative embodiments, the time signal could be inherent in the harvested energy, for example N*60 Hz in inductive charging or lighting.

According to another embodiment, it is possible to have a two-axis system (using either accelerometers or magnetometers). As discussed above, the typical yaw, roll, and pitch description treats all yaw position data as the total yaw. Adding another Euler rotation for each eye (eye yaw) to represent just the eye movement, or eye yaw, and then considering the previously defined yaw, roll, and pitch rotations as common to both eyes, that is, head yaw, head roll and head pitch, thus allows the separation of the eye yaw from the head yaw. Now since the two eye yaw values (θL and θR) are isolated from the rest of the rotational information, the vergence angle may be calculated. The calculation is the difference between the two angles (θL−θR), which is sufficient to compare to the vergence threshold and make a decision, but does not provide any other information. In at least one embodiment, the difference or vergence angle can be determined with just two sensors per eye, X-axis and the Y-axis as shown in FIG. 4B.

In FIG. 4B, a two-axis system controller 411′ controls a lens activator 412 that changes the adaptive optics (see FIG. 5) to control the ability to see both near and far items in focus. The system controller 411′ receives control signals 409′ from a plurality of multidimensional sensors. A first multidimensional sensor includes an X-axis accelerometer or magnetometer 414. A second multidimensional sensor includes a Y-axis accelerometer or magnetometer 415. The system controller 411′ further receives from and supplies signals to communication elements 418. Communication elements 418 allow for communications between the user lens(es) and other devices such as a near-by smartphone. The power source 413 supplies power to all the above system elements. Further functionality of the above embedded elements will be described subsequently. Still further, the system controller 411′ may include the other elements as described with reference to FIG. 2.

While the two-axis system works, a three-axis system provides additional accuracy for situations where there is excess movement, additional lens rotation, and extreme angles. When a sensor axis is perpendicular to the reference vector, the sensor can no longer provide information and thus cannot calculate the vergence angle. In the traditional placement of the accelerometers where the X and Y axes are perpendicular to gravity, two axes are at or very near zero signal or at a null, which can cause the sensor signal to be very low leading to accuracy issues because of noise and other offsets. This issue is very problematic at the normal gaze position since it is looking forward, head straight, but it may be addressed by positioning the sensor such that only one sensor is at a null where the other two are straddling the null and thus have a greater signal, that is not at the null, to improve overall accuracy for the combined sensor system. This is mostly an issue for the accelerometers, because the electromagnetic field of the Earth changes direction and intensity depending on where it is measured.

In at least one embodiment, the addition of the third accelerometer to the system shown in FIG. 4B completes the measurement of gravity such that the root sum of the squares should equal gravity, 9.81 m/s′. Small rotations in the lens can cause errors in the vergence angle calculation; accordingly, the addition of the third axis provides additional information regarding the position of the lens in relationship to the other lens, thus reducing the vergence angle error. This error correction may be employed when using the accelerometer or magnetometer-based system.

Now referring to FIG. 4C, a three-axis system is shown according to an embodiment of the present invention. A system controller 411″ controls a lens activator 412 that changes the adaptive optics (see FIG. 5) to control the ability to see both near and far items in focus. The system controller 411″ receives control signals 409″ from a plurality of multidimensional sensors. A first multidimensional sensor includes an X-axis accelerometer or magnetometer 423. A second multidimensional sensor includes a Y-axis accelerometer or magnetometer 425. A third multidimensional sensor includes a Z-axis accelerometer or magnetometer 427. The system controller 411″ further receives from and supplies signals to communication elements 418. Communication elements 418 allow for communications between the user lens(es) and other devices such as a near-by smartphone. The power source 413 supplies power to the lens located components. Further functionality of the above embedded elements will be described hereafter. Still further, the system controller 411″ may include the other elements as described with reference to FIG. 2.

The accelerometer or magnetometer (423, 425 and 427) measures acceleration both from quick movements and from gravity (9.81 m/s²). The multidimensional sensors (403, 405 and 407) usually produce a value that is in units of gravity (g). The determination of vergence depends on the measurement of gravity to determine position.

Still referring to FIGS. 4A-4C, switching from gaze to accommodation, the system uses the threshold as the activation point. In at least one embodiment, going from accommodation to gaze, the threshold is shifted to a greater distance, thus adding an accommodation threshold hysteresis. Accounting for hysteresis is added in at least one embodiment in order to prevent uncertainty when the user is just at the threshold and there are small head movements which may cause it to switch from gaze to accommodation to gaze, etc. Most likely, the user will be looking at a distant target when the user wants to switch, so the changing of the threshold is acceptable. The hysteresis value may be determined in several ways, for example, the doctor fitting the lenses can change it or the user can change this value via a lens interface.

In today's world, the smartphone is becoming a person's personal communications system, library, payment device, and connection to the world. Applications for the smartphone cover many areas and are widely used. One possible way to interact with the lens(es) in at least one embodiment is to use an application. The application could provide ease of use where written language instructions are used and the user can interact with the app, which provides an interface for the user to receive instructions, information, and feedback and/or provide responses. Voice activation options may also be included as part of the app. For instance, the app may provide the prompting for the sensor calibrations by instructing the user to look forward and prompting the user to acknowledge the process start. The app could provide feedback to the user to improve the calibration and instruct the user what to do if the calibration is not accurate enough for optimal operation. This should enhance the user experience.

Referring now to FIG. 5, shown is still another example implementation according to an exemplary embodiment of the present invention in which sensing and communication may be used to communicate between a pair of contact lenses 505, 507. Pupils 506, 508 are illustrated for viewing objects. The contact lenses 505, 507 include embedded elements 509, 511, such as those illustrated in FIGS. 2 and 4A-4C. The embedded elements 509, 511 include, for example, three-axis accelerometers/magnetometers, lens activators, calibration controller, a system controller, memory, power supply, and communication elements. A communication channel 513 between the two contact lenses 505, 507 allows the embedded elements to conduct calibration between both contact lenses 505, 507. Communication may also take place with an external device, for example, spectacles, a key fob, a dedicated interface device, or a smartphone. Communication between the contact lenses 505, 507 is important to determine proper calibration. Communication between the two contact lenses 505, 507 may take the form of absolute or relative position, or may simply be a calibration signal of one lens to another if there is suspected eye movement. If a given contact lens detects a calibration signal different from the other lens, it may activate a change in stage, for example, switching a variable-focus or variable power optic equipped contact lens to the near distance state to support reading. Other information useful for determining the desire to accommodate (focus near), for example, lid position and ciliary muscle activity, may also be transmitted over the communication channel 513. It should also be appreciated that communication over the channel 513 could include other signals sensed, detected, or determined by the embedded elements 509, 511 used for a variety of purposes, including vision correction or vision enhancement.

The communications channel 513 may include, but is not limited to, a set of radio transceivers, optical transceivers, or ultrasonic transceivers that provide the exchange of information between both lens and between the lenses and a device such as a smart phone, fob, or other device used to send and receive information. The types of information include, but are not limited to, current sensor readings showing position, the results of system controller computation, synchronization of threshold and activation.

Still referring to FIG. 5, the contact lenses 505, 507 further communicate with a smartphone 516 or other external communication device. Specifically, an app 518 on the smartphone 516 communicates with the contact lens(es) 505, 507 via a communication channel 520. The functionally of the app 518 instructs the user when to perform the required eye movements. In addition, the device or smartphone 516 could upload settings, send sequencing signals for the various calibrations, and receive status and error information from the contact lenses 505, 507. It is important to note that any suitable device may be utilized in addition to or instead of the smartphone 516.

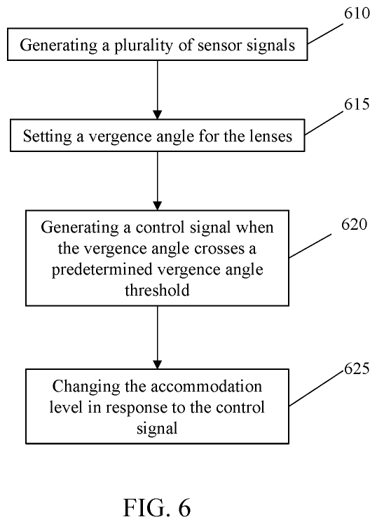

FIG. 6 is a flow chart that illustrates an alternative method for determining vergence angle using two ophthalmic lenses each having a plurality of sensors, a lens activator, and a system controller according to an embodiment of the present invention. The system components are as discussed above. The sensors generate a plurality of sensor signals for at least one of the system controllers in step 610. At least one system controller sets a vergence angle for the lenses based on the plurality of sensor signals from the sensors in step 615. In at least one embodiment, the lenses operate independently in terms of each of in terms of signal generation and vergence angle setting, while in other embodiments there is a dominant system controller that performs the processing. At least one system controller generates a control signal to change accommodation level to the lens activators after the vergence angle has crossed a predetermined vergence angle threshold in step 620. The respective lens activators change the accommodation level of the respective lens in response to the control signal in step 625.

It is important to note that the above described elements may be realized in hardware, in software or in a combination of hardware and software. The various units of the present invention may be embodied within a single processor. In addition, the communication channel may include various forms of wireless communications. The wireless communication channel may be configured for high frequency electromagnetic signals, low frequency electromagnetic signals, visible light signals, infrared light signals, and ultrasonic modulated signals. The wireless channel may further be used to supply power to the internal embedded power source acting as a rechargeable power means.

The present invention may be a system, a method, and/or a computer program product. The computer program product being used by a controller for causing the controller to carry out aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer-readable program instructions.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated.

Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims.

What is claimed is:

1. A user-wearable ophthalmic lens comprising:
a plurality of sensors;
a signal-processing unit in communication with said plurality of sensors and configured to receive sensor signals from said plurality of sensors;
a noise-rejection unit in communication with said signal-processing unit and configured to receive signal-processed signals from said signal-processing unit; and
a decision-making unit in communication with said noise-rejection unit and configured to receive corrected, processed signals from said noise-rejection unit, said decision-making unit configured to change accommodation of the user-wearable ophthalmic lens based on the processed signals.

2. The user-wearable ophthalmic lens according to claim 1, wherein calibration of the at least one ophthalmic lens is initiated upon receipt of a calibration signal from an external device.

3. The user-wearable ophthalmic lens according to claim 2, wherein said plurality of sensors, said signal-processing unit, said noise-rejection unit, and said decision-making unit are configured to determine a customized vergence angle threshold.

4. The user-wearable ophthalmic lens according to claim 2, wherein the external user device is a smartphone.

5. The user-wearable ophthalmic lens according to claim 1, wherein said signal-processing unit, said noise-rejection unit, and said decision-making unit use a customized vergence angle threshold to determine if there is a need to change accommodation.

6. A system comprising:
a pair of ophthalmic lenses, each lens having
a system controller;
a plurality of sensors having a six-axis array to supply sensor signals to said system controller; and
a lens activator configured to receive control signals from said system controller, and
wherein at least one of said system controllers determining a vergence angle for said lenses based on at least signals from said plurality of sensors in said six-axis sensor array per lens and controlling a change in accommodation of at least said lens on which said system controller is located.

7. The system according to claim 6, wherein said system controller in each lens using said plurality of sensors calculates the eye yaw of each eye and then shares the information to calculate the difference of each eye yaw to determine the total vergence angle of the wearer.

8. The system according to claim 6, wherein said six-axis sensor array includes a combination of an accelerometer and magnetometer for X-axis.

9. The system according to claim 8, wherein said six-axis sensor array includes a combination of an accelerometer and magnetometer for Y-axis.

10. The system according to claim 9, wherein said six-axis sensor array includes a combination of an accelerometer and magnetometer for Z-axis.

11. A method for determining vergence angle using two ophthalmic lenses, each having a plurality of sensors, a lens activator, and a system controller, the method comprising:
generating a plurality of sensor signals from the plurality of sensors for at least one of the system controllers;
setting a vergence angle for the lenses by at least one system controller based on the plurality of sensor signals from the plurality of sensors;
generating a control signal to change accommodation level by the at least one system controller for the lens activators after the vergence angle has crossed a predetermined vergence angle threshold; and
changing the accommodation levels of the lenses by the respective lens activator in response to the control signal.

12. The method according to claim 11, wherein the plurality of sensors in each lens is a two-axis sensor array; and
the setting the vergence angle is done by both system controllers using the sensor signals from the respective two-axis sensor array by calculating an eye yaw difference, and the method further comprising sharing the set vergence angle between the system controllers through a communication link.

13. The method according to claim 11, wherein the plurality of sensors in each lens is a two-axis sensor array; and
the setting the vergence angle is done by both system controllers using the sensor signals from the respective two-axis sensor array, and
the method further comprising sharing the set vergence angle between the system controllers through a communication link.

14. The method according to claim 13, wherein the two-axis sensor array includes an accelerometer for an X-axis and a second accelerometer for a Y-axis.

15. The method according to claim 13, wherein the two-axis sensor array includes a magnetometer for an X-axis.

16. The method according to claim 13, wherein the two-axis sensor array includes a magnetometer for a Y-axis.

17. The method according to claim 11, wherein the plurality of sensors in each lens is a three-axis sensor array; and
the setting the vergence angle is done by both system controllers using the sensor signals from the respective three-axis sensor array by calculating an eye yaw difference, and
the method further comprising sharing the set vergence angle between the system controllers through a communication link.

18. The method according to claim 11, wherein the plurality of sensors in each lens is a three-axis sensor array; and
the setting the vergence angle is done by both system controllers using the sensor signals from the respective three-axis sensor array, and
the method further comprising sharing the set vergence angle between the system controllers through a communication link.

19. The method according to claim 11, wherein the plurality of sensors in each lens includes a multi-axis sensor array, and
the method further comprising comparing with the system controller the total signal of each multi-axis sensor array to a known level representing the total acceleration of gravity; and
rejecting the sensor signals when the total signal is out of range.

20. The method according to claim 11, wherein the plurality of sensors in each lens includes a multi-axis sensor array where the axes are offset from measurement nulls such that the measurement axis is perpendicular to a vector representing gravity.
